(19) United States
(12) Patent Application Publication
(10) Pub. No.: US 2026/0248409 A1
(43) Pub. Date: Aug. 27, 2026

(54) PULSE WIDTH MODULATION METHOD AND APPARATUS FOR READING A STRETCHABLE CAPACITIVE SENSOR

(71) Applicant: Tyme Wear, Inc., Somerville, MA (US)

(72) Inventors: Juan Carlos Morales Torrico, Everett, MA (US); Arnar Freyr Larusson, Somerville, MA (US); Arnar S. Hansson, Boston, MA (US); Gregory Laredo, Thornhill (CA)

(73) Assignee: Tyme Wear, Inc., Somerville, MA (US)

(21) Appl. No.: 19/559,689

(22) Filed: Mar. 6, 2026

Related U.S. Application Data
(63) Continuation-in-part of application No. 19/548,924, filed on Feb. 24, 2026.
(63) Continuation-in-part of application No. PCT/US2026/016514, filed on Feb. 24, 2026.
(60) Provisional application No. 63/763,218, filed on Feb. 25, 2025.
(60) Provisional application No. 63/763,218, filed on Feb. 25, 2025.
(60) Provisional application No. 63/768,848, filed on Mar. 7, 2025.

(51) Int. Cl. A61B 5/08; A61B 5/00; A61B 5/113; G01D 5/24; G01D 5/246

(52) U.S. Cl. CPC A61B 5/08; A61B 5/1135; A61B 5/6823; A61B 5/6831; A61B 5/7203; A61B 5/7225; A61B 5/7228; A61B 5/7264; A61B 5/7271; G01D 5/24; G01D 5/246; A61B 2560/0462

(57) ABSTRACT

The techniques described herein relate to a pulse width modulation method and apparatus for reading a stretchable capacitive sensor. An example circuit includes a signal converter configured to convert a capacitance of a stretchable capacitive sensor into a first signal, and control circuitry configured to determine a physiological parameter value for a user based on a rate of change of the first signal.

### CROSS-REFERENCE TO RELATED APPLICATIONS

The present application claims the benefit of U.S. Provisional Patent Application No. 63/768,848, filed Mar. 7, 2025 under Attorney Docket No. T0961.70001US00 and entitled “PULSE WIDTH MODULATION METHOD AND APPARATUS FOR READING A STRETCHABLE CAPACITIVE SENSOR,” which is hereby incorporated by reference in its entirety.

The present application claims the benefit of priority under 35 U.S.C. § 120 and is a continuation-in-part of International Patent Application No. PCT/US2026/016514, filed Feb. 24, 2026 under Attorney Docket No. T0961.70000WO00 and entitled “METHODS, SENSORS, AND SYSTEMS FOR MEASURING VENTILATION THRESHOLDS FOR IMPROVED FITNESS,” which claims the benefit of priority under 35 U.S.C. § 119 of U.S. Provisional Patent Application No. 63/763,218, filed Feb. 25, 2025 under Attorney Docket No. T0961.70000US00 and entitled “METHODS, SENSORS, AND SYSTEMS FOR MEASURING VENTILATION THRESHOLDS FOR IMPROVED FITNESS.”

The present application further claims the benefit of priority under 35 U.S.C. § 120 and is a continuation-in-part of U.S. patent application Ser. No. 19/548,924, filed Feb. 24, 2026 under Attorney Docket No. T0961.70000US01 and entitled “METHODS, SENSORS, AND SYSTEMS FOR MEASURING VENTILATION THRESHOLDS FOR IMPROVED FITNESS,” which claims the benefit of priority under 35 U.S.C. § 119 of U.S. Provisional Patent Application No. 63/763,218, filed Feb. 25, 2025 under Attorney Docket No. T0961.70000US00 and entitled “METHODS, SENSORS, AND SYSTEMS FOR MEASURING VENTILATION THRESHOLDS FOR IMPROVED FITNESS.”

International Patent Application No. PCT/US2026/016514, U.S. patent application Ser. No. 19/548,924, and U.S. Provisional Patent Application No. 63/763,218 are hereby incorporated by reference herein in their entireties.

### FIELD

The techniques described herein relate generally to circuits and, more particularly, to a pulse width modulation method and apparatus for reading a stretchable capacitive sensor.

### BACKGROUND

VO2 max and ventilatory thresholds are physiological parameters that represent the maximum amount of oxygen and the fractional utilization of oxygen that an individual can achieve during exercise. VO2 max and ventilatory thresholds can be measured using sophisticated lab equipment having flow sensors that sense the flow of oxygen and carbon dioxide. Such equipment is expensive and of limited accessibility, making it of limited use to those seeking to assess and improve upon their current fitness level.

If VO2 max is the only physiological parameter sought, then sometimes it is indirectly determined using wearable sensors that measure heart rate, Global Positioning System (GPS) data, or accelerometry. An indirect determination of VO2 max can be derived from heart rate by making various assumptions about the user and then applying known calculations linking the heart rate to VO2 max. However, such calculations deriving VO2 max from heart rate may not provide an accurate assessment of VO2 max, and also do not provide any measure of ventilatory thresholds.

### SUMMARY

In accordance with the disclosed subject matter, a pulse width modulation method and apparatus for reading a stretchable capacitive sensor are provided.

Some embodiments relate to a circuit comprising a signal converter configured to convert a capacitance of a stretchable capacitive sensor into a first signal, and control circuitry configured to determine a physiological parameter value for a user based on a rate of change of the first signal.

Some embodiments relate to another circuit comprising an integrator configured to generate an excited sensor signal at least in part by exciting a variable rate charging device with a pulse width modulation signal, a comparator configured to generate a comparator output signal in response to the excited sensor signal meeting a signal threshold, and a controller configured to determine a physiological parameter value for a user based on a rate of change of the excited sensor signal, the rate of change based at least in part on the comparator output signal.

Some embodiments relate to a method comprising converting a capacitance of a stretchable capacitive sensor into a first signal, and determining a physiological parameter value for a user based on a rate of change of the first signal.

Some embodiments relate to a method comprising exciting a variable rate charging device with a pulse width modulation signal to generate an excited sensor signal, determining a rate of change of the excited sensor signal based at least in part on the excited sensor signal meeting at least one signal threshold, and determining a physiological parameter value for a user based on the rate of change of the excited sensor signal.

Some embodiments relate to an apparatus comprising at least one memory storing processor-executable instructions, and at least one hardware processor configured to execute the processor-executable instructions to perform a method. The method comprising exciting at least one stretchable capacitive sensor with a pulse width modulation signal to generate an excited sensor signal, determining a rate of change of the excited sensor signal based at least in part on the excited sensor signal meeting at least one signal threshold; and determining a value of a physiological parameter for a user based on the rate of change of the excited sensor signal.

Some embodiments relate to at least one computer-readable storage medium storing processor-executable instructions that, when executed by at least one hardware processor, cause the at least one hardware processor to perform a method. The method comprising exciting at least one stretchable capacitive sensor with a pulse width modulation signal to generate an excited sensor signal, determining a rate of change of the excited sensor signal based at least in part on the excited sensor signal meeting at least one signal threshold, and determining a value of a physiological parameter for a user based on the rate of change of the excited sensor signal.

Some embodiments relate to a system for determining a physiological parameter of a user, the system comprising at least one stretchable capacitive sensor having a capacitance that changes in response to physiological changes in the user, at least one hardware processor, and at least one computer-readable storage medium storing processor-executable instructions that, when executed by the at least one hardware processor, cause the at least one hardware processor to perform a method. The method comprising exciting the at least one stretchable capacitive sensor with a pulse width modulation signal to generate an excited sensor signal, determining a rate of change of the excited sensor signal based at least in part on the excited sensor signal meeting at least one signal threshold, and determining a value of a physiological parameter for the user based on the rate of change of the excited sensor signal.

### BRIEF DESCRIPTION OF FIGURES

Various aspects and embodiments of the present technology will be described with reference to the following figures. It should be appreciated that the figures are not necessarily drawn to scale. Items appearing in multiple figures are indicated by the same or a similar reference number in all the figures in which they appear.

FIG. 1A shows an example system including a wearable sensor configured to measure chest wall excursion and in communication with an electronic device, in accordance with some embodiments of the technology described herein.

FIG. 1B is a graph illustrating measured ventilation thresholds and associated intensity zones, in accordance with some embodiments of the technology described herein.

FIG. 2 shows a schematic illustration of another example system including a wearable sensor, processor circuitry including sensor data processing circuitry, and optionally a server, for measuring chest wall excursion, in accordance with some embodiments of the technology described herein.

FIG. 3 is a schematic illustration of an example implementation of the sensor data processing circuitry of FIG. 2, in accordance with some embodiments of the technology described herein.

FIG. 4 shows waveforms depicting example operation of the sensor data processing circuitry of FIGS. 2 and/or 3, in accordance with some embodiments of the technology described herein.

FIGS. 5A-5C show positioning of a strap having one or more wearable sensors on a user, in accordance with some embodiments of the technology described herein.

FIG. 6A shows a stretchable portion and a non-stretchable portion of the wearable sensor of FIGS. 5A-5C, in accordance with some embodiments of the technology described herein.

FIG. 6B shows an example system including a wearable sensor with a single housing, in accordance with some embodiments of the technology described herein.

FIG. 7 is a flowchart representative of an example process that may be performed and/or implemented using hardware logic or machine-readable instructions that may be executed by processor circuitry to implement the sensor data processing circuitry of FIGS. 2 and/or 3 to determine a physiological parameter associated with a user, in accordance with some embodiments of the technology described herein.

FIG. 8 is a flowchart representative of an example process that may be performed and/or implemented using hardware logic or machine-readable instructions that may be executed by processor circuitry to implement the sensor data processing circuitry of FIGS. 2 and/or 3 to output a physiological parameter value based at least in part on a pulse width modulation signal, in accordance with some embodiments of the technology described herein.

FIG. 9 is an example electronic platform structured to execute the machine-readable instructions of FIGS. 7 and/or 8 to implement the sensor data processing circuitry of FIGS. 2 and/or 3, in accordance with some embodiments of the technology described herein.

### DETAILED DESCRIPTION

The present technology generally provides techniques for reading a stretchable capacitive sensor to determine a physiological parameter of a user, such as a VO2 max level or ventilation (ventilatory) threshold, and/or breathing rate of a user's breathing activity. Beneficially, the techniques can be used to determine and provide the physiological parameter to a user for improving the user's fitness and/or, more generally, health and wellbeing.

Aspects of the present technology provide circuits and methods for reading and processing signals from wearable stretchable capacitive sensors. Wearable breathing sensors may comprise a stretchable capacitive sensor which, if placed on the user's chest, stretches and contracts in response to the user's breathing. The sensor may have a variable capacitance that varies based on the sensor's length, such that a variable output signal is produced in response to the user's breathing. The sensor output signal representing the user's chest excursion may be used to determine characteristics of the user's breathing such as the respiration rate and tidal volume. Those parameters allow calculation of minute ventilation, which may be useful in assessing the user's fitness level and/or current state of exertion.

Aspects of the present technology provide circuits for interfacing with stretchable capacitive sensors of the type described to precisely and consistently read an output signal of the sensor to determine the sensor capacitance. The circuits may operate to accurately identify the capacitive output signal. In some embodiments, the circuits include a pulse width modulation (PWM) circuit.

The techniques involve sensor data processing circuitry configured to process a PWM input signal and a signal representative of the sensor's capacitance into a measurable time interval. The sensor data processing circuitry can process the measurable time interval into a quantification of the physiological parameter. Advantageously, the sensor data processing circuitry can leverage the high accuracy, configurability, and linearity of PWM techniques to quantify a user's breathing with enhanced accuracy and reduced power and circuitry footprint.

The inventors recognized that heart rate does not correlate well with ventilatory (ventilation) thresholds that represent an individual's fractional utilization of their VO2 max. For example, while a user's heart rate may increase during exercise, the volume inhaled or exhaled by the user may drop. Moreover, two people can have the same VO2 max but different fractional utilization of their VO2 max. Thus, in many instances using heart rate does not provide an accurate assessment of VO2 max, and furthermore does not provide an accurate assessment of exertion level or fitness level.

According to some embodiments described herein, the fractional utilization of a person's aerobic capacity can be assessed, which may be done by determining ventilatory thresholds. In some embodiments, two or more ventilatory thresholds may be determined from a breathing signal, such as the minute ventilation signal. The aerobic capacity may be represented as VO2 max as described herein. Ventilatory thresholds can determine fractional utilization of aerobic capacity such that breathing uncovers the ventilatory thresholds. Measuring breathing includes both measuring breathing rate and tidal volume, which produces minute ventilation. Minute ventilation responds to changes in muscle substrate utilization. Muscle substrate utilization is determined by the ratio of fat versus carbohydrates (“carbs”) that the muscle is using, wherein fat is efficient and carbs are inefficient. The ratio of fat to carbs determines the efficiency of the work performed. By targeting these transitions of decreasing efficiency in training, the muscles of the individual can adapt by becoming more efficient, thereby increasing fitness level, including improving their ability to go faster for longer. The inventors have recognized that the ventilatory thresholds are the measurable phenomena that exposes these transitions such that they can be measured and targeted in training.

The inventors recognized that the minute ventilation exhibited by an individual across increasing exercise intensity exhibits three inflection points primarily driven by the increasing CO2 production as a result of increasing carbohydrate utilization within the working muscles. One inflection point may be when maximum fat utilization is reached and carbohydrate utilization begins to increase in order to continue to match energy demands of the increasing exercise intensity. Another inflection point may be the aerobic threshold occurring when fat utilization begins to decline causing carbohydrate utilization to increase even more. Another inflection point may be the anaerobic threshold where fat is minimal and carbohydrates make up the entire production which eventually becomes unsustainable. These three inflection points occur in that order as a user increases exercise intensity. That is, as a user increases exercise intensity, the user's minute ventilation will first experience the threshold prompted by maximal fat utilization being reached and carbohydrate utilization beginning to increase, then the aerobic threshold prompted by fat utilization dropping off with a resulting increase in carbohydrate consumption, and then the anaerobic threshold prompted by carbohydrates making up the majority of production which eventually becomes unsustainable.

For purposes of this document, the three thresholds exhibited in the user's ventilation corresponding to increased intensity are referred to as the “low ventilatory threshold,” (or simply the “low threshold”), the “medium ventilatory threshold,” (or simply the “medium threshold”) and the “high ventilatory threshold,” (or simply the “high threshold”) since they appear in that order in the user's ventilation as a function of exercise intensity. That is, the “low ventilatory threshold” (or simply “low threshold”) is the first threshold that occurs at the lowest exercise intensity. The “medium ventilatory threshold” (or simply “medium threshold”) is the next threshold that occurs at increased exercise intensity. The “high ventilatory threshold” (or simply “high threshold”) is the ventilatory threshold that appears at even higher exercise intensity and prior to reaching VO2 max. In some instances, the low ventilatory threshold may result from a physiological change experienced by the user in which maximum fat utilization is reached and carbohydrate utilization begins to increase in order to continue to match energy demands of the increasing exercise intensity. The medium threshold may result from a physiological change at which maximum fat utilization occurs at the aerobic threshold. Between the low and the medium thresholds is sometimes referred to as the “fat plateau.” The high ventilatory threshold may result from a physiological change where lactate production becomes unsustainable at the anaerobic threshold. The user's ventilation as described herein can be tracked with a device which produces a minute ventilation signal. The slope of the minute ventilation signal leading into successive ventilation thresholds (low, medium, and high) will increase for each individual such that for any given individual the slope leading into the medium ventilatory threshold will be higher than the slope leading into the low ventilatory threshold, and the slope leading into the high ventilatory threshold will be higher than the slope leading into the medium ventilatory threshold. Two or more of the low, medium, and high ventilatory thresholds may be used as described herein to analyze the fractional utilization of a person's aerobic capacity.

The low, medium, and high ventilatory thresholds as described above are sometimes called by different names in the field. For example, “Endurance” may be used to refer to the low ventilatory threshold. “VT1” may, at times, be used in the field to refer to the low ventilatory threshold and, at other times, to refer to the medium ventilatory threshold. “VT1” is sometimes also referred to as “the aerobic threshold” or “sweet spot.” “VT2” may, at times, be used in the field to refer to the medium ventilatory threshold and, at other times, to the third ventilatory threshold. “VT2” is sometimes also referred to as the “anaerobic threshold,” “critical power,” or “FTP.”

The techniques described herein may involve measuring the user's ventilation (ventilatory) thresholds using a combination of tidal volume, breathing rate, and minute ventilation data from a wearable sensor. According to the techniques described herein, two or more inflection points in a breathing signal may be determined. The inflection points represent physiological changes as described above.

As described herein, the minute ventilation data can be accurately and consistently determined using a stretchable capacitive sensor fixed to a strap having a fixed initial stretchable length. The user may wear the strap around their chest or, more generally, torso, and the user's breathing may be sensed by the user's chest excursion. The chest excursion signal provides breathing rate and, using sensors of the types described herein, an accurate indication of tidal volume. The minute ventilation can be calculated from the breathing rate and tidal volume, and the user's ventilation thresholds (low, medium, and high as described above, or referred to by names such as VT1, BP or Endurance, and VT2) and VO2 max may be measured manually or using an automated tool such as a machine learning model (e.g., a neural network) trained to identify such inflection points in the ventilation signal. Moreover, measuring one or more ventilatory thresholds may be based on the tidal volume and may comprise measuring the one or more ventilatory thresholds based on a minute ventilation signal. Two of the ventilation thresholds (low, medium, and high as described above, or referred to by names such as VT1, BP, Endurance, or VT2) and/or VO2 max may be used and/or measured in some examples. In some embodiments, the minute ventilation may be calculated using breathing rate times tidal volume. In some embodiments, linear regression, two-line best fit, three-line best fit, four-line best fit, or a Gaussian model that assess break point (looking for 1, 2, or 3 break points) may be used to extract one or more thresholds of a minute ventilation signal.

According to some aspects, the present technology provides techniques that may include segmenting an individual's ventilation response to exercise into four (4) distinct segments. Each segment may be defined by the absolute value of ventilation and the rate of increase within each segment. The transitions between these segments may be referred to as low, medium, and high thresholds as described above, or referred to by names such as VT1, BP or Endurance, VT2, and VO2 max, corresponding to ventilatory thresholds and VO2 max.

The direct measurement of the user's ventilation thresholds (low, medium, and high as described above, or referred to by names such as VT1, BP or Endurance, and VT2), allows for user-specific assessments of fitness to be made, which in turn allows for tailored activity recommendations. In contrast to calculating the ventilation thresholds, for example based solely on population statistics, aspects of the present technology provide increased personalization with improved activity recommendations. Moreover, aspects of the present application provide versatile wearable sensors which may be worn by users in day-to-day activity and exercising situations, thus facilitating regular and low-cost measurement of fitness. Aspects of the present application allow for continuous monitoring of fitness and measurement of ventilation thresholds.

The inventors have further recognized that determining how an individual can improve their fitness is furthered by accurately determining ventilatory threshold(s), which determination relies on an accurate and reliable ventilation signal. Historically, getting an accurate and reliable threshold assessment has been challenging because it either involves lab testing to acquire an accurate ventilation signal combined with manual expert analysis of the data collected or the use of invasive test equipment in the field with blood samples analyzed by a lactate meter to identify appropriate training targets. Non-measurement approaches use formulas that make statistical assumptions applied to a person's activity data or use trend analysis and comparisons of datasets to make predictions or assessments of someone's fitness level. The lab methods and invasive methods are cumbersome, costly, and time consuming. The estimates made by formulas with or without physiological data are prone to high error margins and inconsistent assessments, making them ineffective at assessing fitness level and changes therein or in prescribing training.

The inventors have developed technology that overcomes the above technological challenges. The technology involves attaching a strap to a user to monitor physiological changes in the user. Variable rate charging device(s), such as stretchable capacitive sensor(s), can be fixed to the strap. The strap can have a fixed initial stretchable length. The user may wear the strap around their chest and/or, more generally, torso, and the user's breathing may be sensed by the user's chest excursion. Quantification of the chest excursion can be used to derive respiration rate and, using sensors of the types described herein, an accurate indication of tidal volume. Tidal volume is a physiological parameter representing the amount of air that is inhaled and exhaled from a user's lungs with each respiratory cycle. The tidal volume can be used to derive minute ventilation. The minute ventilation can be used to derive a ventilation threshold.

The stretchable capacitive sensor can be a stretchable sensing element that has a known initial length and measures the expansion and contraction of the chest cavity of a person. The sensor can have a capacitance that changes (e.g., varies) responsive to the stretchable sensing element expanding and contracting with the chest cavity. As the chest walls expand and contract, the stretchable sensing element comes closer together and then goes farther apart, respectively, causing a capacitance change that corresponds to the expansion and contraction of the chest. The sensor can be of any length provided it stretches as the chest expands and contracts. If the sensor is attached to other elements to fasten it around the chest, then these elements must be non-stretchable. In some embodiments, the sensor is a 50 millimeter (mm) long sensor with electronics on one end that receive, store, process, and/or transmit a signal indicative of a physiological parameter for a user.

The sensor can be attached anywhere along the vertical axis of the user's rib cage. For example, the sensor may be attached to a vest, shirt, or strap. The sensor may be permanently attached to the vest, shirt, or strap. The sensor may be detachable to the vest, shirt, or strap via hook and loop (e.g., Velcro), snaps, or temporary adhesive. The sensor, according to some embodiments, is attached between two anchor points on either side of the sensor and when the chest wall expands the two anchor points move further apart and when they contract the two anchor points come back together. More than one sensor may be used.

Sensor data processing circuitry can excite the sensor to generate an excited sensor signal representative of the capacitance (e.g., the instantaneous value of the capacitance) of the sensor. The sensor data processing circuitry can process the excited sensor signal into a physiological parameter for a user using a signal conversion stage, a comparison stage, and a processing stage.

In the signal conversion stage, a PWM generator (e.g., a PWM generation circuit) generates and/or outputs a PWM signal. The PWM input signal can alternate between high and low states. The PWM signal can excite the sensor to generate an excited sensor signal. The excited sensor signal may be a voltage. Alternatively, the excited sensor signal may be an electrical current.

In the signal conversion stage, the PWM signal can excite the signal by charging and/or discharging the sensor, which can be implemented at least in part by one or more capacitors having a variable capacitance. The rate at which the sensor is charged (e.g., via the PWM signal) varies with the change in capacitance of the sensor, such that the excited sensor signal when viewed over time may represent a rate of change of a physiological parameter of a user. The integrator can be configured to process the PWM input signal and the capacitance of the sensor into the excited sensor signal having rising and falling voltage slopes. The rate of these slopes is directly affected by the sensor's capacitance since capacitance influences charging and discharging time of a capacitor, the slopes effectively encode the sensor's capacitance value.

In the comparison stage, the sensor data processing circuitry can measure how long it takes for the excited sensor signal to transition between at least two predefined signal thresholds. The signal thresholds may be voltage thresholds. Alternatively, the signal thresholds may be current thresholds.

The at least two predefined signal thresholds can include a low signal threshold (e.g., a low voltage threshold) and a high signal threshold (e.g., a high voltage threshold). In some embodiments, the comparison stage can be implemented by using two comparators configured to detect when the excited sensor signal crosses these signal thresholds. Each comparator can be configured to trigger an output signal (e.g., a comparator output signal, a high output signal) when its respective signal threshold is met by the excited sensor signal.

In the processing stage, processor circuitry can monitor the comparator outputs and generate timestamps at moments when the excited sensor signal crosses the signal thresholds. The time difference between these two events (e.g., crossing the low signal threshold and crossing the high signal threshold) corresponds to the charging time of a capacitor of the sensor. Since the charging time is proportional to capacitance, this time interval provides a direct measurement of the sensor's capacitance. By sampling (e.g., continuously sampling) this process, the processor circuitry can track changes in the sensor's capacitance and process them in relation to the stretch percentage of the stretchable sensing element. For example, the processor circuitry can determine, generate, and/or output a value of a physiological parameter, such as a VO2 max value (e.g., a VO2 max measurement) or ventilation threshold value (e.g., a ventilation threshold measurement), based at least in part on the stretch percentage as determined based at least in part on the sensor's capacitance.

The techniques described herein may be implemented in any of numerous ways, as the techniques are not limited to any particular manner of implementation. Examples of details of implementation are provided herein solely for illustrative purposes. Furthermore, the techniques disclosed herein may be used individually or in any suitable combination, as aspects of the technology described herein are not limited to the use of any particular technique or combination of techniques.

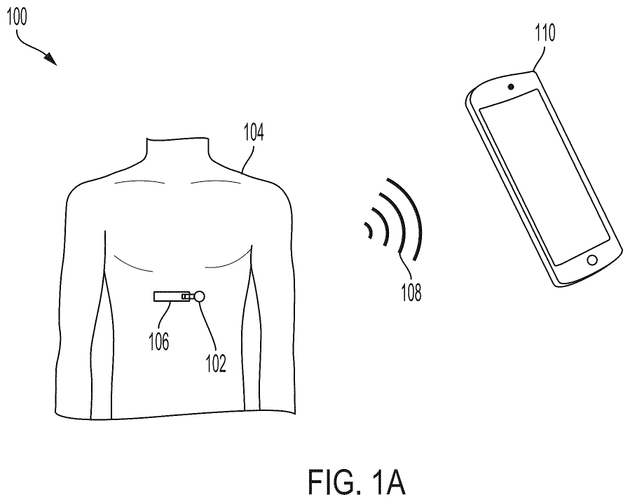

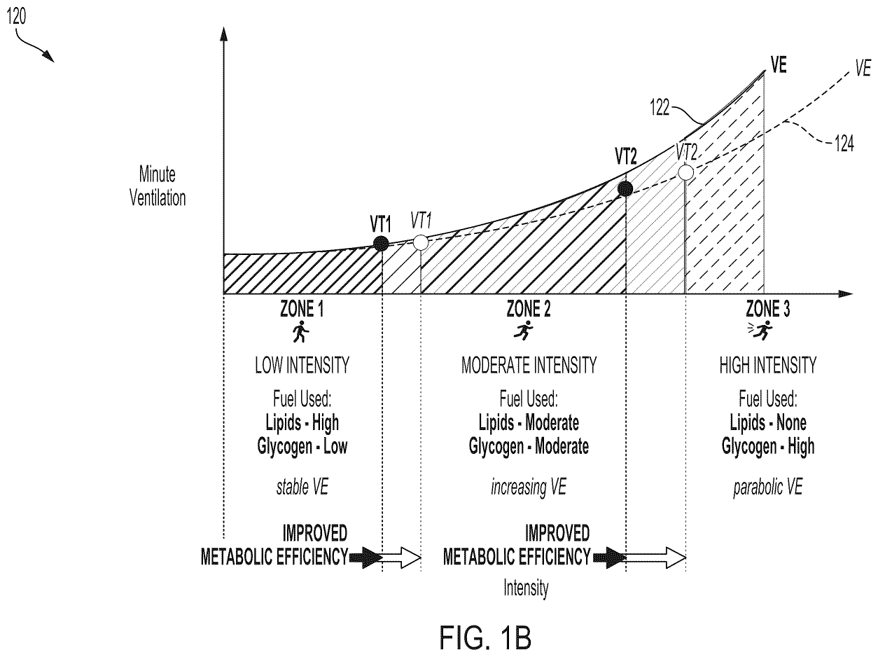

Turning to the figures, the illustrated example of FIG. 1A shows an example system 100 including a wearable sensor 102 configured to measure chest wall excursion of a user 104. As shown, the user 104 is a human person. Alternatively, the user 104 may be an animal.

The wearable sensor 102 is coupled to a strap 106, which is coupled to a chest of the user 104. Alternatively, the wearable sensor 102 may be coupled to the user 104 using an adhesive. Together, the wearable sensor 102 and the strap 106 may form a wearable device and/or, more generally, a wearable system.

The wearable sensor 102 is in communication 108 with an electronic device 110. As shown, the communication 108 is wireless communication. The wireless communication can be a wireless signal. Examples of wireless communication include Bluetooth, near-field communication (NFC), Zigbee, and Wireless Fidelity (Wi-Fi) Direct communication. For example, the wearable sensor 102 may transmit data wirelessly to the electronic device 110 using Bluetooth.

As shown, the electronic device 110 is a cellular phone (e.g., a smartphone).

Alternatively, the electronic device 110 may be a laptop computer, a tablet computer, a television (e.g., smart television), or a different type of wearable device (e.g., headphones, headsets, smartwatches, smart glasses, etc.).

The wearable sensor 102 can be configured to determine a physiological parameter of the user 104, such as a VO2 max value or ventilation threshold value of the user 104. For example, the wearable sensor 102 can include at least one stretchable capacitive sensor having a capacitance (e.g., a variable capacitance) that changes in response to expansion and contraction of the chest cavity of the user 104. In such an example, the wearable sensor 102 can process the capacitance into a measurable time interval. The wearable sensor 102 can process the measurable time interval into a quantification of the physiological parameter of the user 104. For example, the wearable sensor 102 can process a change in capacitance of the sensor into a measurable time interval, which may correspond to a rate of change of the physiological parameter of the user 104.

In some embodiments, processing of both the breathing signal and physiological parameter can happen on a microprocessor physically connected to the wearable sensor 102. The outputs of the processing can be streamed in real-time or saved and sent after the fact to another device, such as the electronic device 110.

FIG. 1B is a graph 120 illustrating measured ventilation thresholds and associated intensity zones. The graph may plot intensity and minute ventilation. The zones may be based in part on known user information including age, gender, and past activities in addition to a current session for the user.

As shown in FIG. 1B, metabolic efficiency can be improved as the ventilation thresholds (VT1 and VT2) increase in intensity in Zone 1 and Zone 2, for example. As discussed herein, the ventilation threshold naming convention may differ, and VT1 and VT2 in FIG. 1B may be referred to as medium and high ventilation thresholds, among other names. Minute ventilation (VE) 122 may be measured at a first time. Minute ventilation (VE) 124 may be measured at a second, later time after a period of training directed to improving metabolic efficiency, such as by using the techniques described herein.

Breathing during exercise is predominantly driven by CO₂ levels in the body. Breathing is driven by inputs including the metabolic demand of a task at hand which drives the need to inhale oxygen and exhale CO₂. The metabolic demand is primarily met via minute ventilation. Stress state of the individual will drive the breathing rate response such that more stress generally increases breathing rate. If stress increases while the metabolic load is held constant, then the tidal volume will decrease proportionally to avoid hyperventilation.

CO₂ is the waste byproduct of cellular respiration as an individual's muscles metabolize their fuel resources, predominantly lipid and glycogen, to meet the energy demand of the work being performed. In fact, when someone loses weight, it is in part because they have breathed out more carbon atoms in the form of CO₂ than the carbon atoms they have retained from the food they've consumed. Understanding how CO₂ drives minute ventilation is key to understanding how breathing gives us a window into individuals' metabolic efficiency.

As exercise intensity goes from low to high, the body traverses three phase shifts driven by its fuel consumption. When energy demand is relatively low, a person's muscles are able to rely in large part on lipids. Lipids produce less volume of CO₂ for each gram of lipid that is consumed as compared to the amount of CO₂ produced by each gram of carbohydrate. This results in lower levels of minute ventilation since the lungs only need to supply enough air to satisfy the O₂ demand. Lipids are both abundant and energy dense. The body stores approximately 2 week's worth of lipids at a given time and lipids produce 2× more energy from one gram compared to 1 gram of glycogen. However, it is relatively harder to extract energy from lipids, and as exercise intensity increases to a moderate level, the body begins to transition to a more readily available fuel source, glycogen. For each gram of glycogen consumed, 30% more CO₂ is produced, and only half the energy is generated compared to lipid consumption. The increased CO₂ production drives higher ventilation to get rid of the excess. This transition is referred to as low ventilation threshold, among other names including the First Ventilatory Threshold (VT1).

As should be appreciated, the ventilation thresholds, shown in FIG. 1B and described elsewhere herein, can have different names, but they are related to physiological changes as described herein. Therefore, while naming conventions may be assigned to ventilatory thresholds herein, the ventilatory thresholds may be named differently in other contexts. The same points, however, are identified independent of the naming conventions. As an example, a first shift during exercise may occur at a first inflection point. The first inflection point may be identified as a first threshold and may be referred to as “VT1” or “Endurance.”

Once the first threshold is traversed and intensity continues to rise, lipid consumption will eventually start to decline. The more metabolically efficient someone is (e.g., higher fitness level), the higher the intensity is at which the lipid consumption declines. The start of the lipid decline may, in some contexts, be referred to as medium ventilation threshold among other names including the Balance Point or, in other contexts, VT1. As lipid consumption declines, it is replaced by sufficient glycogen to match the energy producing capacity of the lipid decline, if workload is increasing, then the energy requirement from the increased workload is met by increased glycogen consumption. The increased glycogen consumption causes ventilation to begin increasing at a greater rate to remove excess CO₂ that is being produced. Each gram of lipid needs to be replaced by 2 grams of glycogen which in turn produces 60% more CO₂. This compounding effect increases CO₂ production and drives ventilation higher, eventually producing a parabolic increase.

As exercise intensity climbs higher, the body eventually transitions away from lipids entirely while increasing its use of glycogen to meet the energy demand of the workload. At this point, the metabolic system goes into a negative feedback loop where fuel consumption has reached unsustainable levels while relying on one of the body's most scarce fuel sources. This transition is called the high ventilation threshold among other names including Second Ventilatory Threshold (VT2).

Beyond this point, the depletion of glycogen stores and/or the acidification from compounding CO₂ levels, and/or build up of H+ ions in the muscles, along with a host of other associated effects of unstable metabolic activity, will eventually contribute to the reduction of the muscle's ability to perform work, forcing the body to slow down. At this point, improvement in a user's efficiency may be achieved given that certain loads are on the user.

In summary, with each transition, glycogen consumption increases and drives CO₂ production and the corresponding increase in minute ventilation to expel it. By measuring breathing, specifically minute ventilation, these transitions can be assessed and targeted in training. The body's ability to delay these transitions is a result of greater ability to consume lipids, and more efficient utilization of glycogen which is referred to as the Glycogen Sparing effect. This phenomenon is achieved by calibrating training intensities and durations around each person's unique transition points. It is the underlying mechanism by which fitness is increased in order to go faster for longer.

Breathing provides an accessible method to assess metabolic output and efficiency. Aspects of the present technology provide for measuring the increase in Minute Ventilation to identify each of the transitions between the four phases, and provides actionable and individualized targets to improve metabolic efficiency. Changes over time can be tracked by how the ventilation response changes.

Low, medium, and high ventilation thresholds, or referred to by names such as VT1, BP, Endurance, or VT2, and/or VO2 max may be visually identified from the breathing signals obtained during a ramp test protocol or obtained using a neural network algorithm that has been trained to identify the inflection points based on labeled datasets (e.g., tens of labeled datasets or hundreds of labeled datasets) where the thresholds have been visually identified by an experienced practitioner. In some embodiments, a model has been trained using data and an algorithm other than a neural network. In some embodiments, the model, including a neural network or other type of model, identifies how a user's breathing pattern changes over time. The model may analyze a signal on a breath-by-breath basis. The model may analyze heart rate (such as in beats per minute) and minute ventilation, as described herein. The trained model may be used to extract one or more thresholds from a minute ventilation signal.

In some embodiments, measuring ventilation thresholds such as low, medium, and high as described above, or referred to by names such as VT1, VT2, BP or Endurance, and VO2 max comprises using fixed benchmarks that have been predetermined for that user, such as a heart rate or ventilation value associated with the user's threshold that was determined in a prior threshold test or one or more workouts. In some embodiments, the fixed benchmarks may be determined using a calibrated data set without a prior threshold test or in combination with data from a prior threshold test.

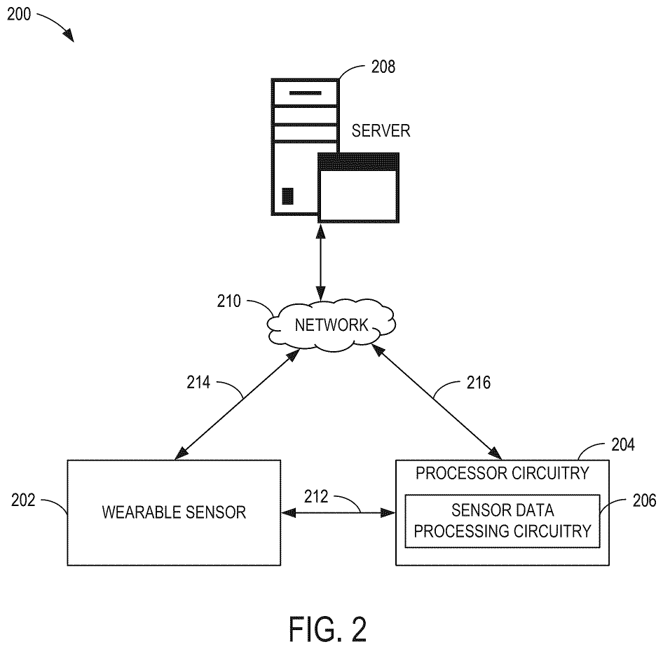

FIG. 2 shows a schematic illustration of another example system 200 including a wearable sensor 202, processor circuitry 204 including sensor data processing circuitry 206, and optionally a server 208, for measuring chest wall excursion of a user, such as the user 104 of FIG. 1A.

In some embodiments, the wearable sensor 202 can correspond to the wearable sensor 102 of FIG. 1A. The wearable sensor 202 of this example can include and/or implement at least one stretchable capacitive sensor configured to have a variable capacitance that changes in response to physiological changes in the user monitored by the wearable sensor 202.

As shown, the wearable sensor 202 is coupled to the processor circuitry 204. For example, the wearable sensor 202 can be coupled via a wired connection to the processor circuitry 204. For example, the wearable sensor 202 can be in communication with the processor circuitry 204 via a bus. In some embodiments, the bus can be any type of computing and/or electrical bus, such as an Inter-Integrated Circuit (I2C) bus, a Peripheral Component Interconnect (PCI) bus, a Peripheral Component Interconnect Express (PCIe) bus, a Serial Peripheral Interface (SPI) bus, a Universal Chiplet Interconnect Express (UCIe) bus, and/or the like.

The wearable sensor 202 can be coupled via a wireless connection to the processor circuitry 204. For example, the wearable sensor 202 can be in communication with the processor circuitry 204 via a Bluetooth, NFC, and/or Wi-Fi Direct connection.

In some embodiments, the processor circuitry 204 can be implemented by one or more programmable processors. Examples of programmable processors include microcontrollers, central processing units (CPUs), digital signal processors (DSPs), field programmable gate arrays (FPGAs), and graphics processing units (GPUs).

The wearable sensor 202 and the processor circuitry 204 may exchange data 212. The processor circuitry 204 may perform processing of electrical output (e.g., the data 212) from the wearable sensor 202 using the sensor data processing circuitry 206. For example, the sensor data processing circuitry 206 may convert a capacitance of the wearable sensor 202 into a sensor signal (e.g., an excited sensor signal). The sensor data processing circuitry 206 may process the sensor signal into a VO2 max value for the user 104 of FIG. 1A.

The sensor data processing circuitry 206 and/or the processor circuitry 204 may store the VO2 max value. For example, the sensor data processing circuitry 206 may store the VO2 max value in at least one datastore accessible to the sensor data processing circuitry 206 and/or, more generally, the processor circuitry 204. In some such embodiments, raw sensor data from the wearable sensor 202 and/or VO2 max values may be stored in non-volatile and/or volatile memory. The non-volatile and/or volatile memory may be included in the sensor data processing circuitry 206 and/or the processor circuitry 204.

As shown, at least one of the wearable sensor 202 or the processor circuitry 204 is in communication with the server 208 via at least one network 210. The wearable sensor 202 may exchange data 214 with the server 208 via the at least one network 210. The processor circuitry 204 may exchange data 216 with the server 208 via the at least one network 210.

The at least one network 210 may be implemented by any wired and/or wireless network(s) such as one or more cellular networks (e.g., 4G LTE cellular networks, 5G cellular networks, future generation 6G cellular networks, etc.), one or more data buses, one or more local area networks (LANs), one or more optical fiber networks, one or more private networks, one or more public networks, one or more satellite networks, one or more wireless local area networks (WLANs), etc., and/or any combination(s) thereof. For example, the at least one network 210 may be the Internet, but any other type of private and/or public network may be used.

The server 208 of this example may perform processing of electrical output from the wearable sensor 202. For example, the server 208 may process a capacitance of the wearable sensor 202 received from the wearable sensor 202 into VO2 max values or ventilation thresholds for the user 104 of FIG. 1A and store the VO2 max values or the ventilation thresholds in at least one datastore accessible to the server 208. The at least one datastore may implement one or more databases. The server 208 may execute, host, and/or implement a service to process and/or store data from the wearable sensor 202 and/or provide processed data to a user.

In some embodiments, the server 208 can be implemented by one or more servers (e.g., computer servers) accessible via a network (e.g., a computer-implemented network). For example, the server 208 can be implemented by one or more physical servers and/or virtualizations of the one or more physical servers. In some embodiments, the one or more servers are hosted by a cloud provider (e.g., a public cloud provider, a private cloud provider) and/or an enterprise network.

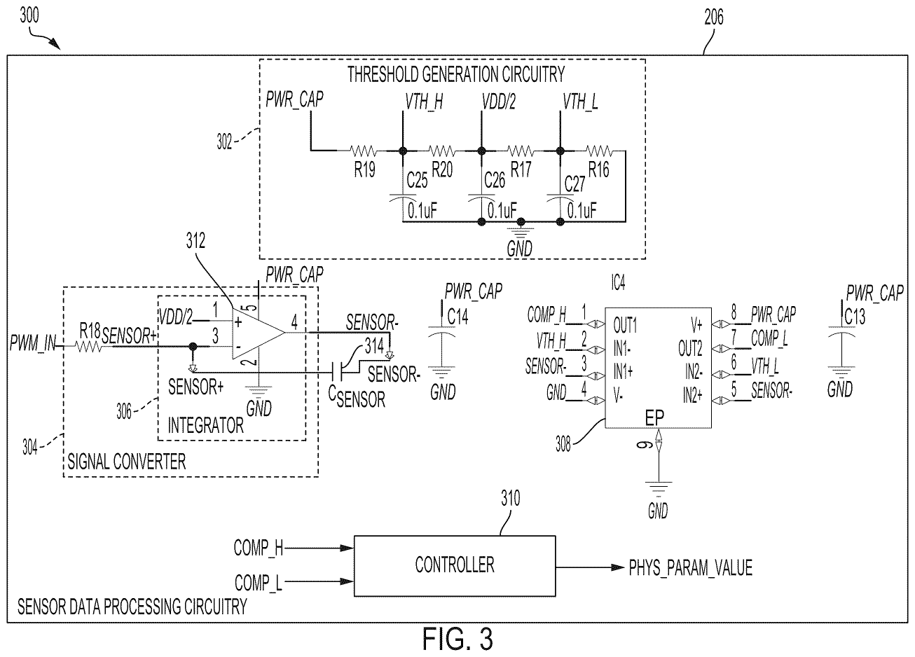

FIG. 3 is a schematic illustration of wearable sensor circuitry 300. In some embodiments, the wearable sensor circuitry 300 can implement and/or correspond to the wearable sensor 102 of FIG. 1A. In some embodiments, the wearable sensor circuitry 300 can implement and/or correspond to the wearable sensor 202 of FIG. 2, the processor circuitry 204 of FIG. 2, the sensor data processing circuitry 206 of FIG. 2, and/or any combination(s) thereof.

As shown, the wearable sensor circuitry 300 includes the sensor data processing circuitry 206 of FIG. 2 and threshold generation circuitry 302. The sensor data processing circuitry 206 shown is an example implementation of the sensor data processing circuitry 206 shown in FIG. 2.

The threshold generation circuitry 302 is configured to generate signals for use by the sensor data processing circuitry 206 to effectuate reading of a capacitive sensor 314. In some embodiments, the wearable sensor 102 of FIG. 1A can correspond to and/or be implemented by the capacitive sensor 314. In some embodiments, the wearable sensor 202 of FIG. 2 can correspond to and/or be implemented by the capacitive sensor 314.

The generated signals may include signal thresholds for the sensor data processing circuitry 206. As shown, the threshold generation circuitry 302 is configured to generate a high signal threshold identified as “VTH_H” and a low signal threshold identified as “VTH_L”. The high and low signal thresholds are voltage thresholds. Alternatively, the threshold generation circuitry 302 may be configured to generate the high and low signal thresholds as current thresholds.

The threshold generation circuitry 302 can be configured to generate VTH_H to be 1.2 volts (V) and VTH_L to be 0.6 V based on an input voltage of 1.8 V with the input voltage identified as “PWR_CAP” and the configuration of the resistors identified by R19, R20, R17, and R16, and the capacitors identified by C25, C26, and C27. Different signal threshold values and/or input voltage values may be used. For example, VTH_L can be determined by the following equation:

$$VTH\_L = ( PWR\_CAP * R 16 ) / ( R 16 + R 17 ) , \qquad Equation ( 1 )$$

And VTH_H can be determined by the following equation:

$$VTH\_H = ( PWR\_CAP * R 20 ) / ( R 19 + R 20 ) , \qquad Equation ( 2 )$$

In some embodiments, R19 and R16 can have the same resistance as each other and R17 and R20 can have the same resistance as each other. For example, R16 and R19 can each have a resistance of 400, 500, 600, etc., kiloohms. In the same or another example, R17 and R20 can each have a resistance of 720, 820, 920, etc., kiloohms. Different resistance values for one or more of the resistors may be used.

In some embodiments, C25, C26, and C27 can have the same capacitance as each other while, in other embodiments, one or more of them may be different from each other. For example, C25, C26, and C27 can each have the same capacitance of 0.1, 0.2, 0.3, etc., microfarads. Different capacitance values for one or more of the capacitors may be used.

As shown, the sensor data processing circuitry 206 includes a signal converter 304, an integrator 306, comparator circuitry 308, and a controller 310. The integrator 306 includes and/or implements an amplifier 312. The amplifier 312 of the sensor data processing circuitry 206 is an operational amplifier (op-amp). Alternatively, more than one amplifier and/or different type(s) of amplifiers may be used. The controller 310 may implement control logic and/or control circuitry.

In some embodiments, the sensor data processing circuitry 206 includes the capacitive sensor 314. In some embodiments, the sensor data processing circuitry 206 does not include the capacitive sensor 314. In some such embodiments, the capacitive sensor 314 is coupled to the sensor data processing circuitry 206 as shown in FIG. 3.

In the illustrated example, the signal converter 304 can be configured to convert a capacitance of the capacitive sensor 314 into a first signal identified by “SENSOR−”. The controller 310 may implement control circuitry configured to determine a physiological parameter value for a user, such as the user 104 of FIG. 1A, based on a rate of change of the first signal.

As shown, the threshold generation circuitry 302 provides a voltage identified by VDD/2 to the sensor data processing circuitry 206 as a first input to amplifier 312. VDD/2 may be a reference voltage. The reference voltage may be a constant voltage. For example, the amplifier 312 and/or, more generally, the integrator 306, may implement an isolator between inputs of the amplifier 312 and the components coupled to the output of the amplifier 312. The isolator can be configured to reduce noise in the signal processing pipeline.

As shown, the signal converter 304 excites the capacitive sensor 314 to generate an excited sensor signal identified by SENSOR−. By way of example, a PWM generator (e.g., a PWM generation circuit) (not shown) generates and/or outputs a PWM signal identified by “PWM_IN”. PWM_IN can excite the capacitive sensor 314 by charging the capacitive sensor 314 when the PWM_IN signal transitions to a high signal state. The rate at which PWM_IN charges the capacitive sensor 314 affects the signal input provided to a second input of the amplifier 312. For example, SENSOR+ can be a PWM signal having rising and falling signal edges whose slopes are affected by a capacitance (identified by “C_{SENSOR}”) of the capacitive sensor 314.

In some embodiments, PWM_IN has a frequency of 100 hertz (Hz), a duty cycle of 50%, and a peak voltage of 1.8 V. Alternatively, PWM_IN may have a different frequency, duty cycle, and/or peak voltage.

The rate at which PWM_IN charges the capacitive sensor 314 is based on the capacitance of the capacitive sensor 314. For example, the capacitive sensor 314 can be a stretchable capacitive sensor. In such an example, the capacitance of the stretchable capacitive sensor can vary between a first capacitance representing a 0% stretch (e.g., a minimum stretch) in the stretchable capacitive sensor and a second capacitance (e.g., a capacitance greater than the first capacitance) representing a 100% stretch (e.g., a maximum stretch) in the stretchable capacitive sensor. In some embodiments, the first capacitance is 450 picofarads and the second capacitance is 900 picofarads. Alternatively, the first and/or second capacitance may be different than 450 and 900 picofarads, respectively. In some such embodiments, PWM_IN charges the capacitive sensor 314 faster (e.g., at a higher rate) when the capacitance is 450 picofarads (e.g., the sensor 314 is 0% stretched) than when the capacitance is 900 picofarads (e.g., the sensor 314 is 100% stretched).

The amplifier 312 can be configured in an arrangement to implement the integrator 306. For example, the amplifier 312 can be configured to amplify the voltage difference between its inputs. As PWM_IN alternates between its high and low states and thereby alternates between charging and not charging the capacitive sensor 314, the amplifier 312 produces a signal (identified by “SENSOR−”) having a rising and falling voltage slope whose slope is affected by the capacitance. The rate of these slopes is directly affected by the capacitance of the capacitive sensor 314. Since capacitance influences the charging and discharging time, the slopes effectively encode the capacitance value of the capacitive sensor 314.

As shown, the signal converter 304 generates and/or outputs the excited sensor signal identified by “SENSOR−”, which is provided and/or output to comparator circuitry 308. For example, the amplifier 312 can be coupled to the comparator circuitry 308 via one or more electrical connections. Examples of electrical connections include pads, traces, wires, and vias.

The comparator circuitry 308 of this example is implemented by an integrated circuit (IC). The comparator circuitry 308 can include and/or implement at least two comparators. For example, inputs IN1+ and IN1− and OUT1 of the comparator circuitry 308 can correspond to the inputs and output of a first comparator implemented by the comparator circuitry 308. Inputs IN2+ and IN2− and OUT2 of the comparator circuitry 308 can correspond to the inputs and output of a second comparator implemented by the comparator circuitry 308.

As shown, the comparator circuitry 308 can compare the output of the amplifier 312 using the two comparators. For example, the first comparator can compare the excited sensor signal SENSOR−(IN1+) to the first voltage threshold VTH_H (IN1−) and the second comparator can compare the excited sensor signal (SENSOR−) to the second voltage threshold VTH_L (IN2−).

By way of example, the first comparator can generate, output, and/or assert a high voltage signal (e.g., a logical high signal corresponding to a digital ‘1’) when the excited sensor signal at least meets VTH_H (e.g., 1.2 V). Alternatively, the first comparator may generate, output, and/or de-assert a low voltage signal (e.g., a logical low signal corresponding to a digital ‘0’) when the excited sensor signal falls below and/or otherwise does not meet VTH_H.

Furthering the example, the second comparator can generate, output, and/or assert a high voltage signal when the excited sensor signal at least meets VTH_L (e.g., 0.6 V). Alternatively, the second comparator may generate, output, and/or de-assert a low voltage signal (e.g., a logical low signal corresponding to a digital ‘0’) when the excited sensor signal falls below and/or otherwise does not meet VTH_L.

The controller 310 can be coupled to the comparator circuitry 308. For example, outputs of the comparators (e.g., OUT1, OUT2) implemented by the comparator circuitry 308 can be coupled to respective inputs of the controller 310.

As shown, the controller 310 can be configured to receive the first comparator output signal COMP_H and the second comparator output signal COMP_L from the comparator circuitry 308. The controller 310 can be configured to generate timestamps based on the output signals. For example, the controller 310 can generate a first timestamp when the COMP_L signal transitions from a low signal state to a high signal state. The controller 310 can generate a second timestamp when the COMP_H signal transitions from a low signal state to a high signal state. The controller 310 can determine a time difference between the first and second timestamps. The controller 310 can determine a value of a physiological parameter based on the time difference. The controller 310 can output a signal (identified by “PHYS_PARAM_VALUE”) representative of the value of the physiological parameter.

While an example implementation of the sensor data processing circuitry 206, the threshold generation circuitry 302, the signal converter 304, the integrator 306, the comparator circuitry 308, the controller 310, the amplifier 312, the capacitive sensor 314, and/or, more generally, the wearable sensor circuitry 300, is depicted in FIG. 3, other implementations are contemplated. For example, one or more blocks, components, functions, etc., of the sensor data processing circuitry 206, the threshold generation circuitry 302, the signal converter 304, the integrator 306, the comparator circuitry 308, the controller 310, the amplifier 312, the capacitive sensor 314, and/or, more generally, the wearable sensor circuitry 300, may be combined or divided in any other way. The sensor data processing circuitry 206, the threshold generation circuitry 302, the signal converter 304, the integrator 306, the comparator circuitry 308, the controller 310, the amplifier 312, the capacitive sensor 314, and/or, more generally, the wearable sensor circuitry 300, of the illustrated example may be implemented by hardware alone, or by a combination of hardware, software, and/or firmware. For example, the sensor data processing circuitry 206, the threshold generation circuitry 302, the signal converter 304, the integrator 306, the comparator circuitry 308, the controller 310, the amplifier 312, the capacitive sensor 314, and/or, more generally, the wearable sensor circuitry 300, may be implemented by one or more analog circuits (e.g., capacitors, comparators, diodes, inductors, operational amplifiers, resistors, transistors, etc.), one or more digital circuits (e.g., logic gates, etc.), one or more hardware-implemented state machines, one or more programmable processors, one or more application specific integrated circuits (ASICs), etc., and/or any combination(s) thereof. The sensor data processing circuitry 206, the threshold generation circuitry 302, the signal converter 304, the integrator 306, the comparator circuitry 308, the controller 310, the amplifier 312, the capacitive sensor 314, and/or, more generally, the wearable sensor circuitry 300, of the illustrated example can be implemented by one or more integrated circuits (ICs) on the same die or one or more ICs on two or more different dies, such as one or more ICs on a first die and one or more ICs on a second die.

In some embodiments, the sensor data processing circuitry 206, the threshold generation circuitry 302, the signal converter 304, the integrator 306, the comparator circuitry 308, the controller 310, the amplifier 312, the capacitive sensor 314, and/or, more generally, the wearable sensor circuitry 300, or portion(s) thereof, may be implemented by a system on a chip or system-on-chip (SoC). An SoC is an integrated circuit design that combines elements of an electronic device onto a single chip instead of using separate components. For example, an SoC may include and/or incorporate within itself one or more programmable processors, input and output (I/O) ports, memory, analog input blocks, analog output blocks, etc., and/or any combination(s) thereof. For example, the sensor data processing circuitry 206, the threshold generation circuitry 302, the signal converter 304, the integrator 306, the comparator circuitry 308, the controller 310, the amplifier 312, the capacitive sensor 314, and/or, more generally, the wearable sensor circuitry 300, may be implemented by a single platform and integrates an entire electronic device (or portion(s) thereof), such as a wearable device, onto the platform.

The example implementation of the wearable sensor circuitry 300 shown in FIG. 3 affords several technological benefits. First, the integrator has superior accuracy and linearity compared to other conventional techniques such as using integrated timer circuits. With the approach shown in FIG. 3, a highly linear relationship between capacitance and voltage is achieved. The high input impedance of the amplifier 312 assists with isolating the capacitive sensor to make it less susceptible to external stray capacitance introduced by surrounding components. Additionally, the implementation shown in FIG. 3 can utilize small capacitances.

A further benefit of the integrator approach is flexibility. For example, by adjusting the PWM parameters of the PWM_IN signal, the wearable sensor circuitry 300 can be adapted to different sensor types such that a wider range of capacitances can be measured. The input PWM frequency can be increased to enhance accuracy or decreased to reduce power consumption.

A further benefit is reduction in necessary printed circuit board (PCB) footprint. The approach shown in FIG. 3 requires fewer external components compared to conventional approaches, and the IC packages can be very compact.

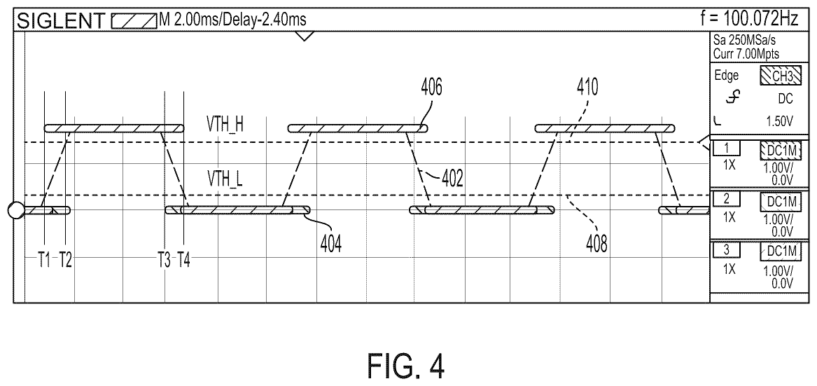

FIG. 4 shows waveforms 402, 404, 406 depicting example operation of the sensor data processing circuitry 206 of FIGS. 2 and/or 3. A first waveform 402 represents the excited sensor signal SENSOR− of FIG. 3. A second waveform 404 represents the comparator output signal COMP_H of FIG. 3. A third waveform 406 represents the comparator output signal COMP_L of FIG. 3. Also shown is VTH_L 408 and VTH_H 410, which correspond to VTH_L and VTH_H of FIG. 3, respectively.

In example operation, the PWM signal PWM_IN of FIG. 3 alternates between its high and low states. When PWM_IN is in the high state it excites the capacitive sensor 314 such that PWM_IN charges the capacitive sensor 314. The rate at which PWM_IN charges the capacitive sensor 314 produces a rising and falling voltage slope of the excited sensor signal 402. The excited sensor signal 402 of this example oscillates as shown between 0 V and 1.8 V. The rate of these slopes is directly affected by the sensor's capacitance. Since capacitance influences the charging and discharging time, the slopes effectively encode the sensor's capacitance value.

In the illustrated example, at a first time (identified by “T1”), when the excited sensor signal 402 increases such that it meets and/or exceeds VTH_L, then the comparator circuitry 308 asserts (e.g., transitions) COMP_L from a low output to a high output and remains asserted until the excited sensor signal 402 falls below COMP_L. At a second time (identified by “T2”), when the excited sensor signal 402 increases such that it meets and/or exceeds VTH_H, then the comparator circuitry 308 asserts COMP_H from a low output to a high output. As shown, COMP_L is asserted and COMP His de-asserted from the first time to the second time.

In example operation, at a third time (identified by “T3”), when the excited sensor signal 402 decreases such that it falls below and/or otherwise does not meet VTH_H, then the comparator circuitry 308 de-asserts COMP_H from a high output to a low output. At a fourth time (identified by “T4”), when the excited sensor signal 402 decreases such that it falls below and/or otherwise does not meet VTH_L, then the comparator circuitry 308 de-asserts COMP_L from a high output to a low output.

As shown, COMP_L and COMP_H are both asserted from the second to the third time. As shown, COMP_L is asserted and COMP_H is de-asserted from the third time to the fourth time. As shown, COMP_L and COMP-H are both de-asserted at the fourth time.

In the illustrated example, the time difference between the rising and falling edges of the excited sensor signal 402 correspond to the charging time of the capacitive sensor 314. Since the charging time is proportional to the capacitance, this time interval provides a direct measurement of the capacitive sensor 314 capacitance. By continuously sampling this process, the wearable sensor circuitry 300 can track changes in the capacitance of the capacitive sensor 314 and interpret them in relation to the stretch percentage of the capacitive sensor 314, which can correspond to the stretch percentage of the wearable sensor 102 of FIG. 1A and/or the wearable sensor 202.

By measuring the time interval between the moments when the excited sensor signal 402 crosses the thresholds VTH_L and VTH_H, the wearable sensor circuitry 300 can accurately approximate the sensor's capacitance in substantially real time. This technique enables precise tracking of capacitance changes corresponding to stretch variations.

In some embodiments, to determine the relative capacitance of the capacitive sensor 314, the time interval between the two comparator-triggered events (e.g., the rising and falling edges of the excited sensor signal 402) is measured. This measurement is performed for both the rising and falling edges of the excited sensor signal 402, and the results are then averaged, for example.

In some embodiments, to enhance accuracy, up to 8 measurements are taken during each full sampling cycle (e.g., a full sampling cycle of 25 Hz), and any outliers are rejected to eliminate anomalies. The final averaged value is then output as a digital reading of the capacitive sensor 314.

By way of example, the controller 310 can receive the comparator output signals COMP_H and COMP_L from the comparator circuitry 308. The controller 310 can be configured with a 16-bit timer and two general purpose input/outputs (GPIOs), COMP_L and COMP_H, set as interrupt triggers. Each event either resets the timer or records a timestamp.

In example operation, in response to detecting a rising edge of COMP_L, the controller 310 can reset the timer and start counting. In response to detecting the rising edge of COMP_H, the controller 310 can stop the timer and record a first time interval (timeInterval1). For example, timeInterval1 can correspond to a time difference between the first time and the second time.

In example operation, in response to detecting a falling edge of COMP_H, the controller 310 can reset the timer and start counting. In response to detecting a falling edge of COMP_L, the controller 310 can stop the timer and record a second time interval (timeInterval2). For example, timeInterval2 can correspond to a time difference between the third time and the fourth time.

In some embodiments, this cycle repeats four times, yielding eight measurements (four rising, four falling), which are stored in a buffer, for example. At the end of the measurement cycle, the data may be filtered by discarding outliers or invalid readings.

In some embodiments, the controller 310 can perform validation checks. Example validation checks include:

If rising COMP_L is detected but rising COMP_H is missing (with only a falling COMP_H present), the reading is discarded. If falling COMP_H is detected following a missing falling COMP_L, the reading is also discarded. If a timer value exceeds the expected limit based on the sensor's maximum capacitance, the reading is discarded. The remaining valid measurements are then averaged to obtain a final result in such an example.

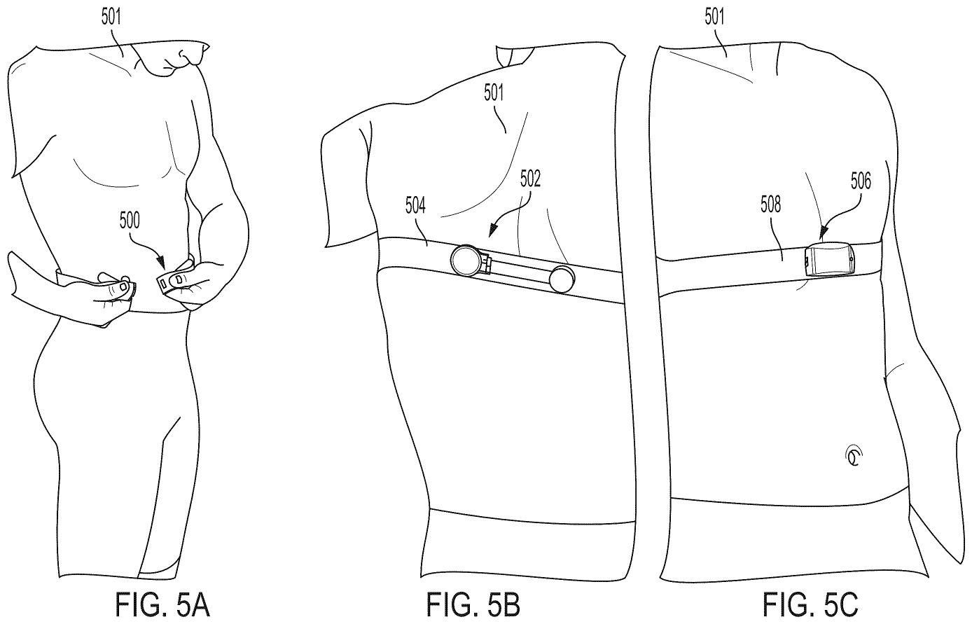

FIGS. 5A-5C show positioning of a strap having one or more wearable sensors on a user 501.

A strap, as described herein, may be implemented as strap 500 as shown in FIG. 5A. A user 501, such as the user 104 of FIG. 1A, may fasten the strap 500 to achieve a snug fit for obtaining measurements as described herein.

Strap 500 may include a stretchable portion and a non-stretchable portion. The stretchable portion 504 may have a capacitive sensor 502 coupled thereto as shown in FIG. 5B. In some embodiments, the capacitive sensor 502 corresponds to the capacitive sensor 314 of FIG. 3.

The capacitive sensor 502 may include a first and second end respectively coupled to the strap 500 as shown. The capacitive sensor 502 may operate to measure chest excursion as described previously herein. The wearable sensor includes a stretchable sensor, and the chest excursion signal is a varying signal representing stretching of the stretchable sensor as the user 501 breathes. The stretchable portion may be correlated to tidal volume. As shown in FIG. 5B, the capacitive sensor 502 may be positioned on a back side of the user 501, although alternative positioning is possible. As shown in FIG. 5B, the capacitive sensor 502 is facing an external surface of the strap 500. The strap 500 may be configured to surround an abdomen or thorax of the user 501, as shown in FIGS. 5A-5C. Strap 500 may include a non-stretchable portion 508 with a sensor 506 coupled thereto as shown in FIG. 5C. The sensor 506 may include a heart rate sensor. As shown in FIG. 5C, the heart rate sensor may be positioned on a front side of the user 501. In other embodiments, the heart rate sensor may be positioned on a back side of the user 501. The heart rate sensor may be configured to obtain heart rate data (such as in beats per minute). The ventilation signal, described elsewhere herein, may be obtained while the strap 500 is positioned on the user 501 to surround an abdomen or thorax of the user 501, such as shown in FIGS. 5B-5C.

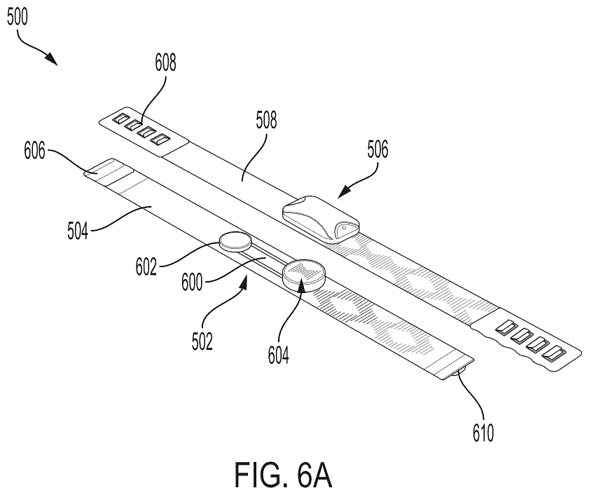

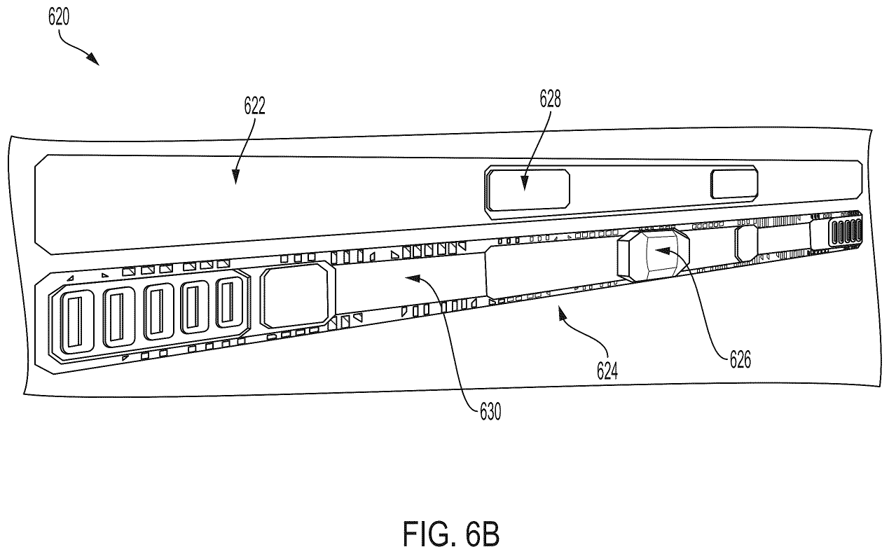

FIG. 6A shows a stretchable and non-stretchable portion of the strap 500 of FIGS. 5A-5C.

As shown in FIG. 6A, capacitive sensor 502 may include a stretchable portion 600 between a first end and second end thereof. The first and second end of the capacitive sensor 502 may be coupled to the strap 500 as shown. As described herein, the capacitive sensor 502 may have a capacitance that changes based at least in part on a length of the stretchable portion of the capacitive sensor 502.

As also shown in FIG. 6A, capacitive sensor 502 may have its first end coupled to a module 604. At the first end of capacitive sensor 502, a first and second electrode may facilitate connection of the capacitive sensor to one or more electronic components of module 604. Module 604 may be configured to house such electronic components, such as the sensor data processing circuitry 206 and/or, more generally, the processor circuitry 204 of FIG. 2, and/or the wearable sensor circuitry 300 of FIG. 3 or portion(s) thereof. The electronic components may include a battery, control circuitry, inertial measurement units (IMUs), accelerometers, and Bluetooth connectivity elements.

In some embodiments, the module 604 may be configured to effectuate signal denoising. For example, the module 604 can include at least one accelerometer and/or at least one IMU configured to measure and/or output acceleration data. The module 604 can filter out motion artifacts from the breathing signal by using the acceleration data. For example, the module 604 can characterize and cancel noise associated with the motion artifacts to improve signal processing.

The second end of the capacitive sensor 502 may be coupled to a connector 602. The second end of the capacitive sensor 502 may have a hole formed therethrough. An adhesive may be applied to the second end where the hole is located for anchoring the second end of the capacitive sensor 502 to the strap 500. The connector 602 may secure the connection to the strap 500 at the second end.

As shown in FIG. 6A, the stretchable and non-stretchable portion of the wearable sensor may include one or more clip mechanisms. The clip mechanisms may couple the stretchable and non-stretchable portions to each other. In the non-limiting example of FIG. 6A, the stretchable portion includes a clip mechanism 606 at each end. The clip mechanisms 606 may have protrusions 610 extending therefrom. The protrusions may be configured to engage with a corresponding clip mechanism 608 on another portion of the wearable sensor.

While some examples of the system described herein (including a wearable sensor) include multiple housings or modules, such as shown in FIG. 6A, the system is not so limited. FIG. 6B shows an example system 620 including a wearable sensor and a single housing 626 for electronics, according to some embodiments. The single housing 626 may house a heart rate measuring unit, a battery, processing unit, control circuitry, IMUs, accelerometers, and Bluetooth connectivity elements. One or more measurements from an inertial measurement unit, an accelerometer, and/or a heart rate sensor coupled to the strap may be obtained. The system 620 may be configured to perform electrocardiogram (ECG) measurements by using at least the heart rate sensor coupled to the strap. For example, the system 620 may be configured to perform an ECG on the user 501.

As shown in FIG. 6B, first and second straps 622 and 624 may be provided that can be coupled together. In some embodiments, additional fit adjustment portions (not shown in FIG. 6B) may be included.

The first strap 622 may include one or more electrodes 628 for heart rate measurements. Second strap 624 may include capacitive sensors 630. Capacitive sensors 630 may have the same material and functionality as capacitive sensor 502 described elsewhere herein. The capacitive sensors 630 may be integrated on stretchable portions of the strap. In some embodiments, the stretchable portions may range from 5 centimeters (cm) to 10 cm each.

As shown in FIG. 6B, two capacitive sensors 630 may be provided on opposite sides of the housing 626. The inventors appreciated that having two capacitive sensors provides redundancy such that if one sensor is not providing accurate readings, the other of the two capacitive sensors may be used. As an example, one of the two capacitive sensors may be prevented from stretching due to an external force, such as from a user laying on their side or sitting on a bike with a seat contacting a portion of the strap, and therefore may not provide useful readings. Further, if both sensors are providing sensor data, the sensor output may be more accurate with two sets of measurements as opposed to one set of measurements. The signals corresponding to the two or more sets of measurements may be combined with averaging or another weighting scheme and/or by summing the two signals to merge them into one signal.

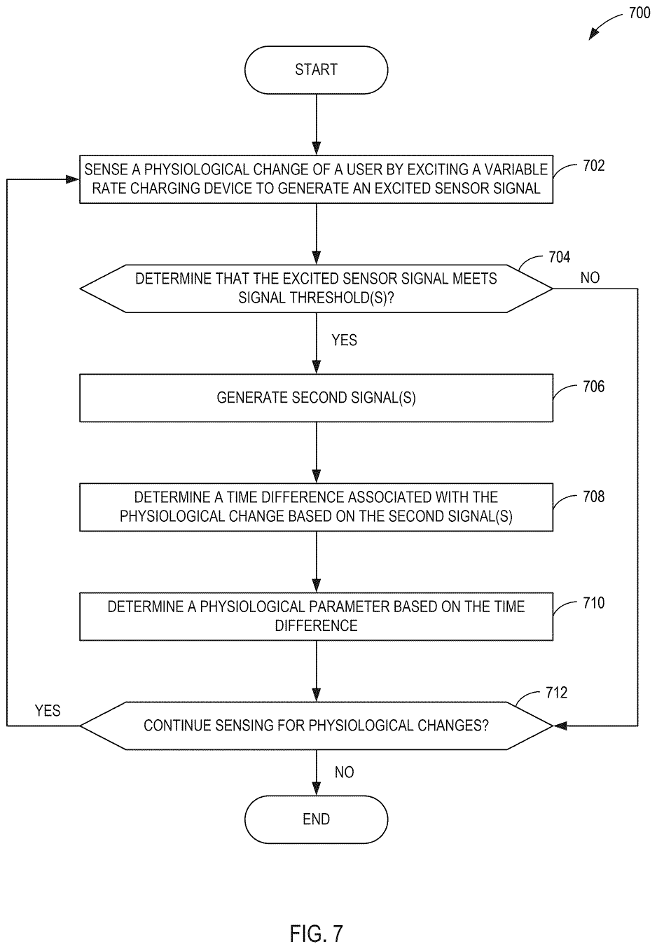

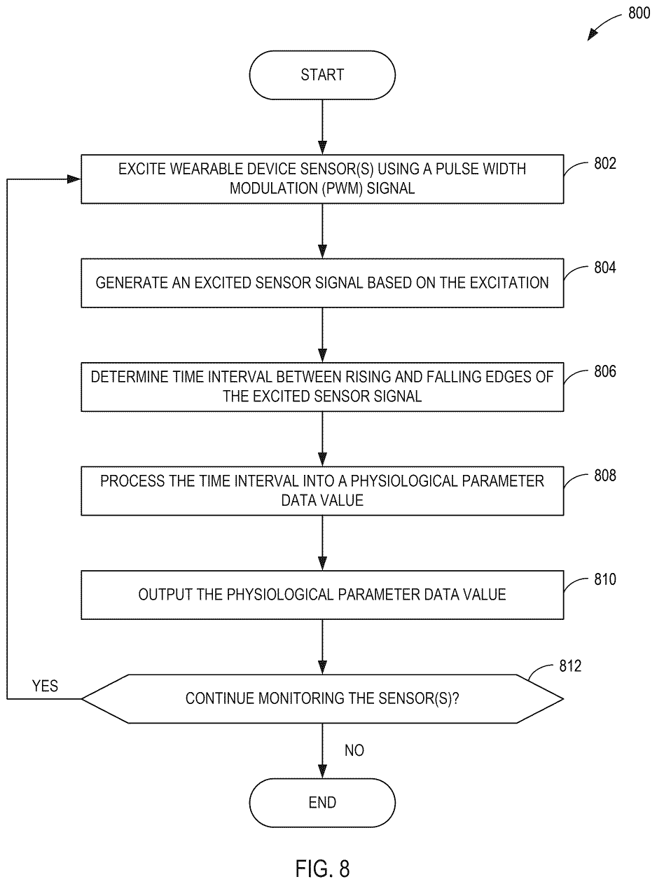

FIGS. 7-8 are flowcharts 700, 800 representative of example processes to be performed and/or example machine-readable instructions that may be executed by processor circuitry to implement the sensor data processing circuitry 206 and/or, more generally, the processor circuitry 204 of FIG. 2. Additionally or alternatively, block(s) of one(s) of the flowcharts 700, 800 of FIGS. 7 and/or 8 may be representative of state(s) of one or more hardware-implemented state machines, algorithm(s) that may be implemented by hardware alone such as an ASIC, etc., and/or any combination(s) thereof.

FIG. 7 is a flowchart 700 representative of an example process that may be performed, example machine-readable instructions that may be executed by processor circuitry, and/or hardware logic to implement the sensor data processing circuitry 206 and/or, more generally, the processor circuitry 204 of FIG. 2 to determine a physiological parameter associated with a user.

The flowchart 700 of FIG. 7 begins at block 702, at which the processor circuitry 204 may sense a physiological change of a user by exciting a variable rate charging device to generate an excited sensor signal. For example, the signal converter 304 can excite the capacitive sensor 314 using input PWM signal PWM_IN to generate and/or output the excited sense signal SENSOR−. The rate of change of the excited sensor signal can correspond to the physiological change of the user.

At block 704, the processor circuitry 204 may determine whether the excited sensor signal meets one or more signal thresholds. For example, the comparator circuitry 308 may compare, by using a first comparator, SENSOR− to VTH_L to determine whether SENSOR− is below VTH_L and therefore does not meet VTH_L or whether SENSOR− is at or exceeds VTH_L and therefore meets VTH_L. Furthering the example, the comparator circuitry 308 may compare, by using a second comparator, SENSOR− to VTH_H to determine whether SENSOR− is below VTH_H and therefore does not meet VTH_H or whether SENSOR− is at or exceeds VTH_H and therefore meets VTH_H.

If, at block 704, the processor circuitry 204 determines that the excited sensor signal does not meet at least one signal threshold, control proceeds to block 712. Otherwise, control proceeds to block 706.

At block 706, the processor circuitry 204 may generate second signal(s). For example, the first comparator may output a high output signal as COMP_L of FIG. 3 when VTH_L is at least met and/or the second comparator may output a high output signal as COMP_H of FIG. 3 when VTH His at least met.

At block 708, the processor circuitry 204 may determine a time difference associated with the physiological change based on the second signal(s). For example, the controller 310 may determine a time difference between a rising and falling edge of the excited sensor signal 402 based on timestamps generated in response to the COMP_L and COMP_H signals being asserted. In such an example, the controller 310 may determine a time difference between the rising and falling edges of the excited sensor signal 402 shown in FIG. 4.

At block 710, the processor circuitry 204 may determine a physiological parameter based on the time difference. For example, the controller 310 may determine the output signal PHYS_PARAM_VALUE of FIG. 3 based on the time difference between the rising and falling edges of the excited sensor signal 402 shown in FIG. 4. PHYS_PARAM_VALUE may be a value of a VO2 max parameter or a ventilation threshold. For example, PHYS_PARAM_VALUE may be a value indicative of a low ventilation threshold, a medium ventilation threshold, or a high ventilation threshold as described elsewhere.

At block 712, the processor circuitry 204 may determine whether to continue sensing for physiological changes. If, at block 712, the processor circuitry 204 determines to continue sensing for physiological changes, control returns to block 702. Otherwise, the flowchart 700 of FIG. 7 concludes.

FIG. 8 is a flowchart 800 representative of an example process that may be performed, example machine-readable instructions that may be executed by processor circuitry, and/or hardware logic to implement the sensor data processing circuitry 206 and/or, more generally, the processor circuitry 204 of FIG. 2 to output a physiological parameter value based at least in part on a pulse width modulation signal.

At block 802, the processor circuitry 204 may excite wearable device sensor(s) using a pulse width modulation (PWM) signal. For example, the signal converter 304 may excite the capacitive sensor 314 using input PWM signal PWM_IN.

At block 804, the processor circuitry 204 may generate an excited sensor signal based on the excitation. For example, the signal converter 304 may process and/or convert the capacitance of the capacitive sensor 314 into the excited sensor signal SENSOR− as shown in FIG. 3.

At block 806, the processor circuitry 204 may determine a time interval between rising and falling edges of the excited sensor signal. For example, the controller 310 may determine the time interval between (i) an average of timestamps T1 and T2 of FIG. 4 and (ii) and average of timestamps T3 and T4 of FIG. 4.

At block 808, the processor circuitry 204 may process the time interval into a physiological parameter data value. For example, the controller 310 may process the time interval into a VO2 max value or a ventilation threshold value for a person, such as the user 104 of FIG. 1A.

At block 810, the processor circuitry 204 may output the physiological parameter data value. For example, the controller 310 may output PHYS_PARAM_VALUE, which can be a VO2 max value or a ventilation threshold value, for display on at least one display device to the user 104, transmit the VO2 max value or the ventilation threshold value to the server 208, etc., and/or any combination(s) thereof.

At block 812, the processor circuitry 204 may continue monitoring the sensor(s). If, at block 812, the processor circuitry 204 determines to continue monitoring the sensor(s), control returns to block 802. Otherwise, the flowchart 800 of FIG. 8 concludes.

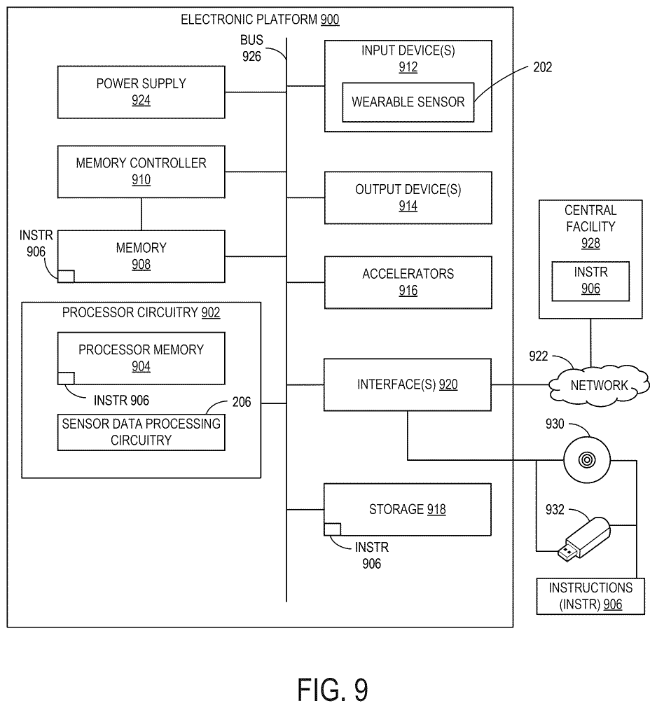

FIG. 9 is an example implementation of an electronic platform 900 structured to execute the machine-readable instructions of FIGS. 7 and/or 8 to implement a wearable device, such as the wearable sensor 102 of FIG. 1A, the wearable sensor 202 and/or the processor circuitry 204 of FIG. 2, and/or the wearable sensor circuitry 300 of FIG. 3. It should be appreciated that FIG. 9 is intended neither to be a description of necessary components for an electronic and/or computing device to operate as a wearable sensor or wearable device, in accordance with the techniques described herein, nor a comprehensive depiction.

The electronic platform 900 of this example may be an electronic device, such as a handset device (e.g., a cellular network device, a smartphone, etc.), a wearable device (e.g., a stretchable capacitive sensor, an augmented reality and/or virtual reality (AR/VR) device, a heads-up display (HUD) device, a fitness tracker, a smartwatch, smart glasses, smart goggles, a medical device patch, a medical bracelet, etc.), or any other type of computing and/or electronic device.

The electronic platform 900 of the illustrated example includes processor circuitry 902, which may be implemented by one or more programmable processors, one or more hardware-implemented state machines, one or more ASICs, etc., and/or any combination(s) thereof. For example, the one or more programmable processors may include one or more CPUs, one or more DSPs, one or more FPGAs, one or more GPUs, etc., and/or any combination(s) thereof. The processor circuitry 902 may include at least one hardware processor (e.g., at least one computer hardware processor). The processor circuitry 902 includes processor memory 904, which may be volatile memory, such as random-access memory (RAM) of any type. The processor circuitry 902 of this example implements the sensor data processing circuitry 206 of FIG. 2 and/or, more generally, the processor circuitry 204 of FIG. 2. For example, the processor circuitry 902 may implement the wearable sensor circuitry 300 of FIG. 3.

The processor circuitry 902 may execute machine-readable instructions 906 (identified by “INSTRUCTIONS”), which are stored in the processor memory 904, to implement the sensor data processing circuitry 206 FIG. 2 and/or, more generally, the wearable sensor 202 of FIG. 2. The machine-readable instructions 906 may be processor-executable instructions that, when executed by the processor circuitry 902, cause the processor circuitry 902 to perform at least one method as described herein. The machine-readable instructions 906 may include data representative of computer-executable and/or machine-executable instructions implementing techniques that operate according to the techniques described herein. For example, the machine-readable instructions 906 may include data (e.g., code, embedded software (e.g., firmware), software, etc.) representative of the flowcharts 700, 800 of FIGS. 7 and/or 8, or portion(s) thereof.

The electronic platform 900 includes memory 908, which may include the instructions 906. The memory 908 of this example may be controlled by a memory controller 910. For example, the memory controller 910 may control reads, writes, and/or, more generally, access(es) to the memory 908 by other component(s) of the electronic platform 900. The memory 908 of this example may be implemented by volatile memory, non-volatile memory, etc., and/or any combination(s) thereof. For example, the volatile memory may include static random-access memory (SRAM), dynamic random-access memory (DRAM), cache memory (e.g., Level 1 (L1) cache memory, Level 2 (L2) cache memory, Level 3 (L3) cache memory, etc.), etc., and/or any combination(s) thereof. In some examples, the non-volatile memory may include Flash memory, electrically erasable programmable read-only memory (EEPROM), magnetoresistive random-access memory (MRAM), ferroelectric random-access memory (FeRAM, F-RAM, or FRAM), etc., and/or any combination(s) thereof.

The electronic platform 900 includes input device(s) 912 to enable data and/or commands to be entered into the processor circuitry 902. For example, the input device(s) 912 may include an audio sensor, a camera (e.g., a still camera, a video camera, etc.), a keyboard, a microphone, a mouse, a touchscreen, a voice recognition system, etc., and/or any combination(s) thereof.

The electronic platform 900 includes output device(s) 914 to convey, display, and/or present information to a user (e.g., a human user, a machine user, etc.). For example, the output device(s) 914 may include one or more display devices, speakers, etc. The one or more display devices may include an augmented reality (AR) and/or virtual reality (VR) display, a liquid crystal display (LCD), a light-emitting diode (LED) display, an organic light-emitting diode (OLED) display, a quantum dot (QLED) display, a thin-film transistor (TFT) LCD, a touchscreen, etc., and/or any combination(s) thereof. The output device(s) 914 can be used, among other things, to generate, launch, and/or present a user interface. For example, the user interface may be generated and/or implemented by the output device(s) 914 for visual presentation of output and speakers or other sound generating devices for audible presentation of output.

The electronic platform 900 includes accelerators 916, which are hardware devices to which the processor circuitry 902 may offload compute tasks to accelerate their processing. For example, the accelerators 916 may include artificial intelligence/machine-learning (AI/ML) processors, ASICs, FPGAs, graphics processing units (GPUs), neural network (NN) processors, systems-on-chip (SoCs), vision processing units (VPUs), etc., and/or any combination(s) thereof. In some examples, the sensor data processing circuitry 206 may be implemented by one(s) of the accelerators 916 instead of the processor circuitry 902. In some examples, the sensor data processing circuitry 206 may be executed concurrently (e.g., in parallel, substantially in parallel, etc.) by the processor circuitry 902 and the accelerators 916. For example, the processor circuitry 902 and one(s) of the accelerators 916 may execute in parallel function(s) corresponding to the sensor data processing circuitry 206.

The electronic platform 900 includes storage 918 to record and/or control access to data, such as the machine-readable instructions 906. The storage 918 may be implemented by one or more mass storage disks or devices, such as HDDs, SSDs, etc., and/or any combination(s) thereof.

The electronic platform 900 includes interface(s) 920 to effectuate exchange of data with external devices (e.g., computing and/or electronic devices of any kind) via a network 922. The interface(s) 920 of the illustrated example may be implemented by an interface device, such as network interface circuitry (e.g., a NIC, a smart NIC, etc.), a gateway, a router, a switch, etc., and/or any combination(s) thereof. The interface(s) 920 may implement any type of communication interface, such as Wi-Fi Direct, NFC, BLUETOOTH®, a cellular telephone system (e.g., a 4G LTE interface, a 5G interface, a future generation 6G interface, etc.), an Ethernet interface, a near-field communication (NFC) interface, an optical disc interface (e.g., a Blu-ray disc drive, a Compact Disk (CD) drive, a Digital Versatile Disk (DVD) drive, etc.), an optical fiber interface, a satellite interface (e.g., a BLOS satellite interface, a LOS satellite interface, etc.), a Universal Serial Bus (USB) interface (e.g., USB Type-A, USB Type-B, USB TYPE-C™ or USB-C™, etc.), etc., and/or any combination(s) thereof.

The electronic platform 900 includes a power supply 924 to store energy and provide power to components of the electronic platform 900. The power supply 924 may be implemented by a power converter, such as an alternating current-to-direct-current (AC/DC) power converter, a direct current-to-direct current (DC/DC) power converter, etc., and/or any combination(s) thereof. For example, the power supply 924 may be powered by an external power source, such as an alternating current (AC) power source (e.g., an electrical grid), a direct current (DC) power source (e.g., a battery, a battery backup system, etc.), etc., and the power supply 924 may convert the AC input or the DC input into a suitable voltage for use by the electronic platform 900. In some examples, the power supply 924 may be a limited duration power source, such as a battery (e.g., a rechargeable battery such as a lithium-ion battery).

Component(s) of the electronic platform 900 may be in communication with one(s) of each other via a bus 926. For example, the bus 926 may be any type of computing and/or electrical bus, such as an I2C bus, a PCI bus, a PCIe bus, a SPI bus, a UCIe bus, and/or the like.

The network 922 may be implemented by any wired and/or wireless network(s) such as one or more cellular networks (e.g., 4G LTE cellular networks, 5G cellular networks, future generation 6G cellular networks, etc.), one or more data buses, one or more local area networks (LANs), one or more optical fiber networks, one or more private networks, one or more public networks, one or more wireless local area networks (WLANs), etc., and/or any combination(s) thereof. For example, the network 922 may be the Internet, but any other type of private and/or public network is contemplated.

The network 922 of the illustrated example facilitates communication between the interface(s) 920 and a central facility 928. The central facility 928 in this example may be an entity associated with one or more servers, such as one or more physical hardware servers and/or virtualizations of the one or more physical hardware servers. For example, the central facility 928 may be implemented by a public cloud provider, a private cloud provider, etc., and/or any combination(s) thereof. In this example, the central facility 928 may compile, generate, update, etc., the machine-readable instructions 906 and store the machine-readable instructions 906 for access (e.g., download) via the network 922. For example, the electronic platform 900 may transmit a request, via the interface(s) 920, to the central facility 928 for the machine-readable instructions 906 and receive the machine-readable instructions 906 from the central facility 928 via the network 922 in response to the request.

Additionally, or alternatively, the interface(s) 920 may receive the machine-readable instructions 906 via non-transitory machine-readable storage media, such as an optical disc 930 (e.g., a Blu-ray disc, a CD, a DVD, etc.) or any other type of removable non-transitory machine-readable storage media such as a USB drive 932. For example, the optical disc 930 and/or the USB drive 932 may store the machine-readable instructions 906 thereon and provide the machine-readable instructions 906 to the electronic platform 900 via the interface(s) 920.

Techniques operating according to the principles described herein may be implemented in any suitable manner. The processing and decision blocks of the flowcharts above represent steps and acts that may be included in algorithms that carry out these various processes. Algorithms derived from these processes may be implemented as software integrated with and directing the operation of one or more single- or multi-purpose processors, may be implemented as functionally equivalent circuits such as a DSP circuit or an ASIC, or may be implemented in any other suitable manner. It should be appreciated that the flowcharts included herein do not depict the syntax or operation of any particular circuit or of any particular programming language or type of programming language. Rather, the flowcharts illustrate the functional information one skilled in the art may use to fabricate circuits or to implement computer software algorithms to perform the processing of a particular apparatus carrying out the types of techniques described herein. For example, the flowcharts, or portion(s) thereof, may be implemented by hardware alone (e.g., one or more analog or digital circuits, one or more hardware-implemented state machines, etc., and/or any combination(s) thereof) that is configured or structured to carry out the various processes of the flowcharts. In some examples, the flowcharts, or portion(s) thereof, may be implemented by machine-executable instructions (e.g., machine-readable instructions, computer-readable instructions, computer-executable instructions, etc.) that, when executed by one or more single- or multi-purpose processors, carry out the various processes of the flowcharts. It should also be appreciated that, unless otherwise indicated herein, the particular sequence of steps and/or acts described in each flowchart is merely illustrative of the algorithms that may be implemented and can be varied in implementations and embodiments of the principles described herein.

Accordingly, in some embodiments, the techniques described herein may be embodied in machine-executable instructions implemented as software, including as application software, system software, firmware, middleware, embedded code, or any other suitable type of computer code. Such machine-executable instructions may be generated, written, etc., using any of a number of suitable programming languages and/or programming or scripting tools, and also may be compiled as executable machine language code or intermediate code that is executed on a framework, virtual machine, or container.

When techniques described herein are embodied as machine-executable instructions, these machine-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way.

Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc., that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement using the exemplary techniques described herein, and that embodiments are not limited to being implemented in any specific number, division, or type of functional facilities. In some implementations, all functionalities may be implemented in a single functional facility. It should also be appreciated that, in some implementations, some of the functional facilities described herein may be implemented together with or separately from others (e.g., as a single unit or separate units), or some of these functional facilities may not be implemented.

Machine-executable instructions (e.g., processor-executable instructions) implementing the techniques described herein (when implemented as one or more functional facilities or in any other manner) may, in some embodiments, be encoded on one or more computer-readable media, machine-readable media, etc., to provide functionality to the media. Computer-readable media, machine-readable media, etc., include magnetic media such as a hard disk drive, optical media such as a CD or a DVD, a persistent or non-persistent solid-state memory (e.g., Flash memory, Magnetic RAM, etc.), or any other suitable storage media. Such a computer-readable medium, a machine-readable medium, etc., may be implemented in any suitable manner. As used herein, the terms “computer-readable media” (also called “computer-readable storage media”), “computer-readable medium” (also called “computer-readable storage medium”), “machine-readable media” (also called “machine-readable storage media”), and “machine-readable medium” (also called “machine-readable storage medium”) refer to tangible storage media. Tangible storage media are non-transitory and have at least one physical, structural component. In a “computer-readable medium” and “machine-readable medium” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium, a machine-readable medium, etc., may be altered during a recording process.

Further, some techniques described above comprise acts of storing information (e.g., data and/or instructions) in certain ways for use by these techniques. In some implementations of these techniques—such as implementations where the techniques are implemented as machine-executable instructions—the information may be encoded on a computer-readable storage media. Where specific structures are described herein as advantageous formats in which to store this information, these structures may be used to impart a physical organization of the information when encoded on the storage medium. These advantageous structures may then provide functionality to the storage medium by affecting operations of one or more processors interacting with the information; for example, by increasing the efficiency of computer operations performed by the processor(s).

In some, but not all, implementations in which the techniques may be embodied as machine-executable instructions, these instructions may be executed on one or more suitable computing device(s) and/or electronic device(s) operating in any suitable computer and/or electronic system, or one or more computing devices (or one or more processors of one or more computing devices) and/or one or more electronic devices (or one or more processors of one or more electronic devices) may be programmed to execute the machine-executable instructions. A computing device, electronic device, or processor (e.g., processor circuitry) may be programmed to execute instructions when the instructions are stored in a manner accessible to the computing device, electronic device, or processor, such as in a data store (e.g., an on-chip cache or instruction register, a computer-readable storage medium and/or a machine-readable storage medium accessible via a bus, a computer-readable storage medium and/or a machine-readable storage medium accessible via one or more networks and accessible by the device/processor, etc.). Functional facilities comprising these machine-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more FPGAs for carrying out the techniques described herein, or any other suitable system.

Embodiments have been described where the techniques are implemented in circuitry and/or machine-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The phrase “and/or,” as used herein in the specification and in the claims, should be understood to mean “either or both,” of the elements so conjoined, e.g., elements that are conjunctively present in some cases and disjunctively present in other cases. Multiple elements listed with “and/or” should be construed in the same fashion, e.g., “one or more” of the elements so conjoined. Other elements may optionally be present other than the elements specifically identified by the “and/or” clause, whether related or unrelated to those elements specifically identified.

The indefinite articles “a” and “an,” as used herein in the specification and in the claims, unless clearly indicated to the contrary, should be understood to mean “at least one.”

As used herein in the specification and in the claims, the phrase, “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

All definitions, as defined and used herein, should be understood to control over dictionary definitions, definitions in documents incorporated by reference, and/or ordinary meanings of the defined terms.

The word “exemplary” is used herein to mean serving as an example, instance, or illustration. Any embodiment, implementation, process, feature, etc., described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

1. A circuit comprising:
a signal converter configured to convert a capacitance of a stretchable capacitive sensor into a first signal; and
control circuitry configured to determine a physiological parameter value for a user based on a rate of change of the first signal.

2. The circuit of claim 1, wherein the first signal is a current or a voltage.

3. The circuit of claim 1, wherein the signal converter comprises an isolator configured to reduce noise associated with one or more inputs of the signal converter.

4. The circuit of claim 1, further comprising comparator circuitry configured to determine the rate of change of the first signal.

5. The circuit of claim 1, wherein the signal converter is configured to convert the capacitance into the first signal as output using an excited sensor signal as input, the excited sensor signal generated by exciting the stretchable capacitive sensor with a pulse width modulation signal.

6. The circuit of claim 5, wherein the signal converter comprises an amplifier configured to generate the first signal as output by amplifying a voltage difference as input, the voltage difference between a voltage of the excited sensor signal and a reference voltage.

7. A circuit comprising:
an integrator configured to generate an excited sensor signal at least in part by exciting a variable rate charging device with a pulse width modulation signal;
a comparator configured to generate a comparator output signal in response to the excited sensor signal meeting a signal threshold; and
a controller configured to determine a physiological parameter value for a user based on a rate of change of the excited sensor signal, the rate of change based at least in part on the comparator output signal.

8. The circuit of claim 7, wherein the variable rate charging device is a stretchable capacitive sensor.

9. The circuit of claim 7, wherein the physiological parameter value represents a VO2 max measurement or a ventilation threshold value for the user.

10. The circuit of claim 7, wherein the physiological parameter value is a value representative of chest wall excursion of the user.

11. The circuit of claim 10, wherein a change in the chest wall excursion of the user causes a change in a capacitance of the variable rate charging device, and the excited sensor signal is representative of the change in the capacitance of the variable rate charging device.

12. The circuit of claim 7, wherein the integrator comprises an amplifier configured to output the excited sensor signal.

13. (canceled)

14. The circuit of claim 7, wherein the signal threshold is a first voltage threshold, the comparator is a first comparator, and further comprising a second comparator configured to:
generate a second comparator output signal in response to the excited sensor signal meeting a second voltage threshold, and wherein
the controller is configured to determine the physiological parameter value based on the rate of change of the excited sensor signal, the rate of change based at least in part on the first comparator output signal and the second comparator output signal.

15. The circuit of claim 14, wherein the controller is configured to:
determine a time difference between a first timestamp at which the first comparator output signal is generated and a second timestamp at which the second comparator output signal is generated; and
determine the rate of change of the excited sensor signal based on the time difference.

16. The circuit of claim 7, wherein a wearable device comprises the circuit, the wearable device comprising:
a strap coupled to the variable rate charging device, the variable rate charging device comprising:
a first end coupled to the strap;
a second end coupled to the strap; and
a stretchable portion between the first end and the second end.

17-36. (canceled)

37. A system for determining a physiological parameter of a user, the system comprising:
at least one stretchable capacitive sensor having a capacitance that changes in response to physiological changes in the user;
at least one hardware processor; and
at least one computer-readable storage medium storing processor-executable instructions that, when executed by the at least one hardware processor, cause the at least one hardware processor to perform a method comprising:
exciting the at least one stretchable capacitive sensor with a pulse width modulation signal to generate an excited sensor signal;
determining a rate of change of the excited sensor signal based at least in part on the excited sensor signal meeting at least one signal threshold; and
determining a value of a physiological parameter for the user based on the rate of change of the excited sensor signal.

38. The system of claim 37, wherein the determining the rate of change of the excited sensor signal is based on a change in a capacitance of the at least one stretchable capacitive sensor.

39. The system of claim 38, wherein the physiological change is a chest wall excursion of the user, a change in the chest wall excursion causes the change in the capacitance of the capacitive sensor, and the excited sensor signal is representative of the change in the chest wall excursion.

40. The system of claim 37, wherein the at least one signal threshold comprises a first voltage threshold and a second voltage threshold, and further comprising:
in response to determining that the excited sensor signal meets the first voltage threshold, generate a first timestamp;
in response to determining that the excited sensor signal meets the second voltage threshold, generate a second timestamp;
determining a time difference between the first timestamp and the second timestamp, and wherein
determining the rate of change is based on the time difference.

41. The system of claim 37, wherein the physiological parameter value represents a VO2 max measurement or a ventilation threshold value for the user.
